(19) United States
(12) Patent Application Publication
(10) Pub. No.: US 2026/0009003 A1
(43) Pub. Date: Jan. 8, 2026

(54) MANUFACTURING OF THERAPEUTIC SATELLITE CELLS FOR TREATING MUSCULAR DYSTROPHY

(71) Applicant: Vita Therapeutics, Inc., Baltimore, MD (US)

(72) Inventors: Congshan SUN, Baltimore, MD (US); Amir SABERI, Baltimore, MD (US); Peter Andersen, Baltimore, MD (US); Douglas FALK, Baltimore, MD (US); Yongming REN, Baltimore, MD (US); Sheela JACOB, Baltimore, MD (US); Jeff HARDING, Baltimore, MD (US); Amy WESA, Baltimore, MD (US)

(21) Appl. No.: 18/993,492

(22) PCT Filed: Jul. 11, 2023
(86) PCT No.: PCT/US2023/027331

§ 371 Date: Jan. 10, 2025

Related U.S. Application Data
(60) Provisional application No. 63/388,187, filed on Jul. 11, 2022.

(51) Int. Cl. C12N 5/077; A61K 35/34; C12N 5/10; C12N 15/85

(52) U.S. Cl. CPC C12N 5/0659; A61K 35/34; C12N 5/10; C12N 15/85; C12N 2506/45; C12N 2510/00; C12N 2533/52; C12N 2830/008

(57) ABSTRACT

The present disclosure provides compositions and methods for preparing autologous satellite cells that have restored a dysfunctional gene involved in muscular dystrophy of a patient. Such implanted satellite cells can promote repair and recovery of myofibrils and muscles in the patients, thereby treating muscular dystrophy in the patient.

### CROSS REFERENCE TO RELATED APPLICATIONS

This application claims the benefit under 35 U.S.C. § 119(e) of the U.S. Provisional Application Ser. No. 63/388,187, filed Jul. 11, 2022, the content of which is hereby incorporated by reference in its entirety.

### BACKGROUND

Limb-girdle muscular dystrophy Type 2A (LGMD2A) is an autosomal recessive LGMD syndrome caused by mutations of the Calpain-3 (CAPN3) gene, which codes a non-lysosomal Ca²⁺ protease and plays a key role in sarcomere remodeling. CAPN3 mutations result in muscle cells that are unable to function appropriately and repair damage that occurs through normal physiologic activity.

There are no drugs or biological products specifically indicated for use in LGMD, or LGMD2A, with no disease-modifying treatments available. In humans, satellite cells are the engine of muscle regeneration and homeostasis. Upon muscle injury, quiescent satellite cells are activated and released from the basal lamina and undergo asymmetric cell division to give rise to one identical satellite cell and one myoblast. The newly formed satellite cell returns to the basal lamina and becomes quiescent, while the newly formed myoblast multiplies in numbers and either repairs the damaged myofiber(s) by fusing with it or it undergoes fusion with other myoblasts to give rise to new myofibers.

In patients with LGMD2A, myoblasts are defective, resulting in the continued decline in muscular structure and function. In addition, the proliferating capacity of LGMD2A patients' satellite cells is severely reduced and overall muscle regeneration is impaired.

### SUMMARY

It is contemplated that muscular dystrophy characterized with a mutant disease gene can be treated with intra-muscular (IM) transplantation of satellite cells that are not deficient in the disease gene, such as autologous satellite cells in which the disease gene has been corrected. Such implanted satellite cells can promote repair and recovery of myofibrils and muscles in the patients.

Accordingly, in one embodiment, the present disclosure provides autologous cell products designed to restore normal expression of the disease gene in satellite cells and their progeny. These satellite cells not only replace and repair damaged muscle tissues but also, upon homing to the satellite cell niche in a quiescent state, support long lasting homeostasis and repair of future muscle damages. The provided treatment to muscles can promote ambulation, mobility, and improve patient's functional status and ability to perform activities of daily living.

Also provided, in one embodiment, are methods for preparing autologous satellite cells with restored gene expression and activity. In an example embodiment the preparation method entails, a) acquiring peripheral blood mononuclear cells (PBMC) from a muscular dystrophy patient; b) reprogramming the PBMC into induced pluripotent stem cells (iPSC); c) correcting the mutant gene in the iPSC; c) differentiating the iPSC into satellite cells; and d) enriching and expanding the satellite cells.

According one embodiment of the present disclosure, provided is a method for preparing a satellite cell from a stem cell comprising a mutation in an endogenous gene. In some embodiments, the method comprises introducing to the stem cell a first and a second vectors, each comprising a first expression cassette encoding at least part of the gene without the mutation, such that each of the first expression cassettes is integrated at a locus of the endogenous gene in the genome to enable coding of a wild-type version of the gene without the mutation; differentiating the stem cell into a satellite cell; and expanding the satellite cell into a population of satellite cells.

Also provided is a method for preparing a satellite cell from a stem cell comprising a mutation in an endogenous gene, comprising: introducing to the stem cell a first and a second vectors, each comprising a first expression cassette comprising all coding sequences of the wild-type gene, such that each of the first expression cassettes is integrated at a locus in the genome different from the endogenous gene; differentiating the stem cell into a satellite cell; and expanding the satellite cell into a population of satellite cells.

In some embodiments, the locus is in a genomic safe harbor, such as those selected from the group consisting of an intron of the AAVS1 gene, and human genome 195,338,589- 195,818,588, 22,720,711-22,761,389, 145,090,941-145,219,513, 145,320,384-145,525,881, and 89,174,426-89,179,074 (GRCh38 coordinates). In some embodiments, the locus is in intron 1 of the AAVS1 gene.

In some embodiments, the expression cassette further comprises a promoter capable to initiating expression of the coding sequences in a muscle cell. In some embodiments, the promoter is a muscle creatine kinase promoter (tMCK).

The mutation may be a homozygous mutation or a heterozygous mutation, without limitation.

In some embodiments, the stem cell is an induced pluripotent stem cell (iPSC) derived from a peripheral blood mononuclear cell or fibroblast isolated from a patient having muscular dystrophy.

In some embodiments, the muscular dystrophy is selected from the group consisting of limb-girdle muscular dystrophy, Becker muscular dystrophy, congenital muscular dystrophy, Duchenne muscular dystrophy, distal muscular dystrophy, Emery-Dreifuss muscular dystrophy, facioscapulohumeral muscular dystrophy, myotonic muscular dystrophy, and oculopharyngeal muscular dystrophy.

In some embodiments, the gene is selected from the group consisting of CAPN3, DNAJB6, TNPO3, HNRPDL, CAPN3, COL6A1, COL6A2, COL6A3, DYSF, SGCA, SGCB, SGCG, SGCD, TCAP, TRIM32, FKRP, TTN, POMT1, ANO5, FKTN, POMT2, POMGNT1, DAG1, PLEC1, TRAPPC11, GMPPB, ISPD, POGLUT1, COL6A1, COL6A2, COL6A3, LAMA2, POMGNT2, POPDC1, POPDC3, JAG2, PYROXD1, DMD, LAMA-2, DMD, DYSF, DMPK, CNBP, DUX4, PABPN1, EMD, LMNA,, SYNE1, SYNE2, FHL1, and TMEM43.

In some embodiments, the gene is CAPN3. For CAPN3, in some embodiments, the at least part of the wild-type CAPN3 gene does not include exon 1 or 2, and the locus is downstream of exon 2 of the endogenous CAPN3 gene. In some embodiments, the at least part of the wild-type CAPN3 gene comprises exons 3-24, and the locus is 3′ to the last nucleotide of exon 2 of the endogenous CAPN3 gene.

In some embodiments, the first vector further comprises a second expression cassette encoding a first selection marker, and the second vector further comprises a third expression cassette encoding a second selection marker, wherein the first expression cassette and the second expression cassette on the first vector together are integrated into the genome, and the first expression cassette and the third expression cassette on the second vector together are integrated into the genome.

In some embodiments, the second and third expression cassettes are excised from the genome following confirmation of the integration. In some embodiments, the first vector and second vector each further comprises a fourth expression cassette encoding a kill switch, which is not integrated to the loci.

In some embodiments, the method further comprises, prior to differentiation, culturing the stem cell in a vessel coated with an adhesion molecule. In some embodiments, the adhesion molecule is laminin-511 or a fragment thereof. In some embodiments, the culturing is in a media comprising a fibroblast growth factor (FGF), a Rho kinase inhibitor, and N2 media.

In some embodiments, the differentiation is carried out in a media comprising a Wnt signaling activator. In some embodiments, the differentiation is further carried out, afterwards, in a media comprising a Notch signaling inhibitor. In some embodiments, the differentiation is further carried out, afterwards, in a media comprising a Notch signaling inhibitor and a TGF-β inhibitor.

In some embodiments, the satellite cell is identified with an antibody specific to CD271. In some embodiments, the identified satellite cell is expanded in a media comprising bFGF2, FGF8b and N2 media.

In some embodiments, the method further comprises administering the prepared satellite cell to a patient having the mutation.

### BRIEF DESCRIPTION OF THE DRAWINGS

FIG. 1 shows an example process work-flow for preparing genetically corrected satellite cells and the use therefore for treating LGMD.

FIG. 2 illustrates certain steps in correcting the mutant CAPN3 genes in a target iPSC.

FIG. 3A-B illustrate the organization of two genetic strategies for integrating correct CAPN3. The first scheme (FIG. 3A) illustrates vectors for integrating correct CAPN3 coding sequences to both alleles of the CAPN3 locus. In the upper vector, from the left to the right, coding sequences include thymidine kinase (TK), CAPN3 and a puromycin resistance gene (Puro^{R}). Both the TK and Puro^{R} are preceded by a constitutive promoter, and each coding sequence also is followed by a poly(A) tail. In addition, the CAPN3 and Puro^{R} cassettes together are flanked by CAPN3 homology arms, left arm (LA) and right arm (RA). In the lower vector, the puromycin resistance gene is replaced with a neomycin resistance gene (Neo^{R}). In both vectors, the selection markers (Puro^{R} or Neo^{R}) are flanked by site-specific recombinase target sites (loxP) to allow removal of these markers after successful integration and confirmation. The second scheme (FIG. 3B) illustrates vectors for integrating correct CAPN3 coding sequences to the AAVS1 locus driven by a TMCK promoter. Overall design is same as described above. From left to the right, coding sequences include thymidine kinase (TK), in both vectors, the selection markers (Puro^{R} or Neo^{R}) are flanked by site-specific recombinase target sites (loxP) to allow removal of these markers after successful integration and confirmation. Both the TK and Puro^{R} or Neo^{R} are preceded by a constitutive promoter, and each coding sequence also is followed by a poly(A) tail. The tMCK promoter CAPN3 and Puro^{R} or Neo^{R} cassettes together are flanked by AAVS1 homology arms, left arm (LA) and right arm (RA).

FIG. 4 illustrates how each of the CAPN3 correction strategies of FIG. 2 leads to production of functional CAPN3 protein.

FIG. 5A-D show the design of PCR strategies (5A and 5C) for confirming the correct insertion of the exogenous coding sequence, and the testing results with such strategies (5B and 5D).

FIG. 6 presents a charts showing the relative expression levels of functional CAPN3 mRNA in myofibers from a healthy donor (control) and two patients (P1 and P2) before and after the insertion.

FIG. 7 shows Western blots confirming the expression of functional CAPN3 protein in in myofibers from a healthy donor (control) and two patients (P1 and P2) before and after the insertion.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, “stem cell” defines a cell with the ability to divide for indefinite periods in culture and give rise to specialized cells. A stem cell may be totipotent, pluripotent, multipotent, oligopotent or unipotent. Non-limiting examples of types of stem cells include somatic (adult) stem cells, embryonic stem cells, parthenogenetic stem cells and/or induced pluripotent stem cells (iPS cells or iPSCs). As used herein, the term “pluripotent stem cells” refers to cells that are: (i) capable of indefinite proliferation in vitro in an undifferentiated state; (ii) maintain a normal karyotype through prolonged culture; and (iii) maintain the potential to differentiate to derivatives of all three embryonic germ layers (endoderm, mesoderm, and ectoderm) even after prolonged culture. Non-limiting examples of currently available pluripotent stem cells include embryonic stem cells and iPSCs. In some embodiments, the stem cells are iPSCs, in particular human iPSCs.

By “muscle stem cell” is meant a self-renewing mononucleate cell that produces as progeny mononucleate myoblasts, which are committed to form multinucleate myofibers via intercellular fusion. Encompassed herein, are muscle stem cells that produce skeletal muscle, smooth muscle, or cardiac muscle.

The term “muscle cell” as used herein refers to any cell which contributes to muscle tissue. Myoblasts, satellite cells, myotubes, and myofibril tissues are all included in the term “muscle cells” and may all be treated using the methods of the invention. Muscle cell effects may be induced within skeletal, cardiac and smooth muscles. Muscle tissue in adult vertebrates will regenerate from reserve myoblasts called “satellite cells”. Satellite cells are distributed throughout muscle tissue and are mitotically quiescent in the absence of injury or disease. Following muscle injury or during recovery from disease, satellite cells will reenter the cell cycle, proliferate and 1) enter existing muscle fibers or 2) undergo differentiation into multinucleate myotubes which form new muscle fiber. The myoblasts ultimately yield replacement muscle fibers or fuse into existing muscle fibers, thereby increasing fiber girth by the synthesis of contractile apparatus components. This process is illustrated, for example, by the nearly complete regeneration which occurs in mammals following induced muscle fiber degeneration; the muscle progenitor cells proliferate and fuse together regenerating muscle fibers.

“Myogenic” cells as described herein are those cells that are related to the origin of muscle cells or fibers. Various molecular markers are known to be specific for the middle and late stages of myogenic differentiation. For example, in C2C12 cells, myosin and Desmins mark the late stages of myogenesis and are largely restricted to myotubes, whereas myogenin and MRF4 mark the middle stages of myogenesis and are found in all myotubes and in many committed myoblasts.

As used herein “satellite cells,” or “myosatellite cells,” refers to small multipotent cells with little cytoplasm found in mature muscle. Satellite cells are precursors to skeletal muscle cells, able to give rise to satellite cells or myoblasts, which give rise to skeletal muscle cells. They have the potential to provide additional myonuclei to their parent muscle fiber, or return to a quiescent state. Upon activation, satellite cells can re-enter the cell cycle to proliferate and differentiate into myoblasts. Satellite cells may exhibit one or more features which may be shared with endogenous satellite cells, including, but not limited to, capacity to repopulate the satellite cell niche, ability to drive muscle regeneration, exhibit appropriate expression of gene markers, appropriate expression of glycoproteins, and expandability in culture.

The terms “subject,” “patient,” “individual,” etc. are not intended to be limiting and can be generally interchanged. That is, an individual described as a “patient” does not necessarily have a given disease, but may be merely seeking medical advice.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” etc. refer to the amount of an agent that is sufficient to achieve a desired effect, as described herein. In embodiments, the term “effective” when referring to an amount of cells or a therapeutic compound may refer to a quantity of the cells or the compound that is sufficient to yield an improvement or a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to an amount of one or more compounds that is sufficient to result in or promote the production of members of the desired cell population, especially compared to culture conditions that lack the one or more compounds.

### Preparation of Genetically Corrected Satellite Cells

The instant inventors have shown that muscular dystrophy characterized with a mutant disease gene can be treated with intra-muscular (IM) transplantation of satellite cells that are not deficient in the disease gene, such as autologous satellite cells in which the disease gene has been corrected. Such implanted satellite cells can promote repair and recovery of myofibrils and muscles in the patients.

Accordingly, in one embodiment, the present disclosure provides autologous cell products designed to restore normal expression of the disease gene in satellite cells and their progeny. These satellite cells not only replace and repair damaged muscle tissues but also, upon homing to the satellite cell niche in a quiescent state, support long lasting homeostasis and repair of future muscle damages. The provided treatment to muscles can promote ambulation, mobility, and improve patients' functional status and ability to perform activities of daily living.

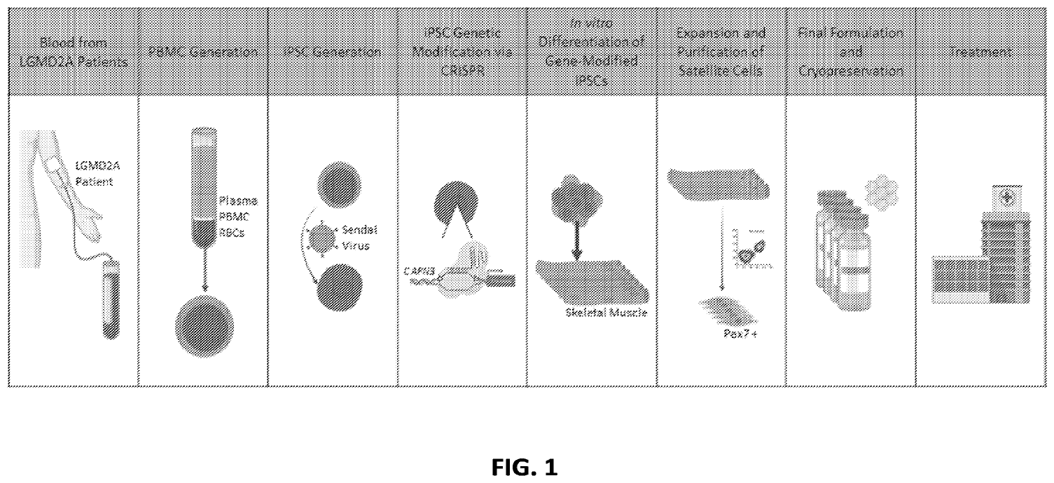

Also provided, in one embodiment, are methods for preparing autologous satellite cells with restored expression and activity of a mutant disease gene. In an example embodiment and with reference to FIG. 1, the preparation method entails:
a) acquiring peripheral blood mononuclear cells (PBMC) from a muscular dystrophy patient characterized with a mutant gene;
b) reprogramming the PBMC into induced pluripotent stem cells (iPSC);
c) correcting the mutant gene in the iPSC;
c) differentiating the iPSC into satellite cells; and
d) enriching and expanding the satellite cells.

It is important to note that not every step is required in the method, and the order of these steps can be adjusted. For instance, the mutant gene may be corrected prior to the PBMC reprogramming. In another example, the iPSC may be expanded prior to differentiation.

Each of these steps is described in more detail below.

### PBMC Acquisition and Reprogramming

Methods of obtaining PBMC are known in the art. For instance, PBMC can be generated from 21 CFR Part 1271 Subpart C compliant patient venous blood using density gradient centrifugation (Steps 1 and 2 in FIG. 1).

The PBMC can be reprogrammed into induced pluripotent stem cells (iPSC) by introducing to the PBMC factors that drive the iPSC formation. Non-limiting examples of such factors include Yamanaka factors Oct 3/4, Sox-2, Klf-4, and L-Myc (Step 3 in FIG. 1). In some embodiments, these factors are transduced to the PMBC with a viral vector, such as the Sendai viral (SeV) vector.

Upon transduction, in some embodiments, the cells are monitored daily, and iPSC colonies are observed after 14 days. It is also important to confirm negative test result with the residual viral assay for two consecutive passages to ensure patient safety.

### Mutant Gene Correction

Mutant gene correction can be carried out with known gene editing technologies. The gene being target depends on the muscular dystrophy treated. Table 1 lists some of the known mutant genes involved in various types of muscular dystrophy.

TABLE 1
Muscular dystrophy types and mutant genes

| | Also | | Mutant |
|---|---|---|---|
| Disease | known as | OMIM | Gene(s) |
| LGMD D1 DNAJB6-related | LGMD1D | 603511 | DNAJB6 |
| LGMD D2 TNP03-related | LGMD1F | 608423 | TNPO3 |
| LGMD D3 HNRNPDL-related | LGMD1G | 609115 | HNRPDL |
| LGMD D4 calpain3-related | LGMD1I | 618129 | CAPN3 |
| LGMD D5 collagen 6-related | Bethlem | 158810 | COL6A1, COL6A2, |
| | myopathy dominant | | COL6A3 |
| LGMD R1 calpain3-related | LGMD2A | 253600 | CAPN3 |
| LGMD R2 dysferlin-related | LGMD2B | 253601 | DYSF |
| LGMD R3 α-sarcoglycan-related | LGMD2D | 608099 | SGCA |
| LGMD R4 β -sarcoglycan-related | LGMD2E | 604286 | SGCB |
| LGMD R5 γ -sarcoglycan-related | LGMD2C | 253700 | SGCG |
| LGMD R6 δ-sarcoglycan-related | LGMD2F | 601287 | SGCD |
| LGMD R7 telethonin-related | LGMD2G | 601954 | TCAP |
| LGMD R8 TRIM 32-related | LGMD2H | 254110 | TRIM32 |
| LGMD R9 FKRP-related | LGMD2I | 607155 | FKRP |
| LGMD R10 titin-related | LGMD2J | 608807 | TTN |
| LGMD R11 POMT1-related | LGMD2K | 609308 | POMT1 |
| LGMD R12 anoctamin5-related | LGMD2L | 611307 | ANO5 |
| LGMD R13 Fukutin-related | LGMD2M | 611588 | FKTN |
| LGMD R14 POMT2-related | LGMD2N | 607439 | POMT2 |
| LGMD R15 POMGnT1-related | LGMD2O | 606822 | POMGNT1 |
| LGMD R16 α-dystroglycan-related | LGMD2P | 613818 | DAG1 |
| LGMD R17 plectin-related | LGMD2Q | 613723 | PLEC1 |
| LGMD R18 TRAPPC11-related | LGMD2S | 615356 | TRAPPC11 |
| LGMD R19 GMPPB-related | LGMD2T | 615352 | GMPPB |
| LGMD R20 ISPD-related | LGMD2U | 616052 | ISPD |
| LGMD R21 POGLUT1-related | LGMD2Z | 617232 | POGLUT1 |
| LGMD R22 collagen 6-related | Bethlem myopathy | | COL6A1, COL6A2, |
| | recessive | | COL6A3 |
| LGMD R23 laminin α2-related | Laminin α2-related | 156225 | LAMA2 |
| | muscular dystrophy | | |
| LGMD R24 POMGNT2-related | POMGNT2-related | 618135 | POMGNT2 |
| | muscular dystrophy | | |
| LGMD R25 | LGMD2X | | POPDC1 |
| LGMD R26 | | | POPDC3 |
| LGMD R27 | | | JAG2 |
| LGMD R(number pending) | | | PYROXD1 |
| Becker muscular dystrophy | | 300376 | DMD |
| Congenital muscular dystrophy | | | LAMA-2 |
| Duchenne muscular dystrophy | | 310200 | DMD |
| Distal muscular dystrophy | | 254130 | DYSF |
| Myotonic muscular dystrophy | | 160900 | DMPK |
| | | 602668 | CNBP |
| Facioscapulohumeral muscular dystrophy | | 158900 | DUX4 |
| Oculopharyngeal muscular dystrophy | | 164300 | PABPN1 |
| Emery-Dreifuss muscular dystrophy 1 | EDMD1 | 310300 | EMD |
| Emery-Dreifuss muscular dystrophy 2 | EDMD2 | 181350 | LMNA |
| Emery-Dreifuss muscular dystrophy 3 | EDMD3 | 616516 | |
| Emery-Dreifuss muscular dystrophy 4 | EDMD4 | 612998 | SYNE1 |
| Emery-Dreifuss muscular dystrophy 5 | EDMD5 | 612999 | SYNE2 |
| Emery-Dreifuss muscular dystrophy 6 | EDMD6 | 300696 | FHL1 |
| Emery-Dreifuss muscular dystrophy 7 | EDMD7 | 614302 | TMEM43 |

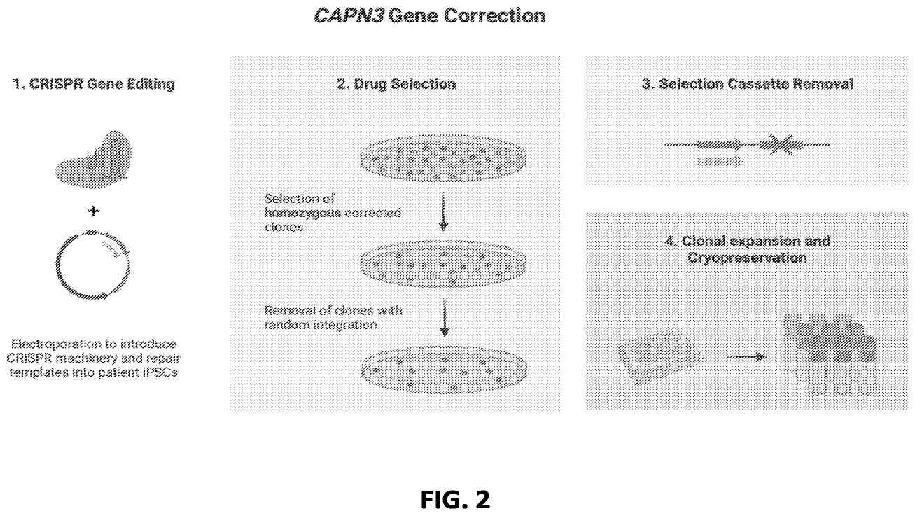

The following description uses CAPN3 as an example mutant gene in muscular dystrophy (FIG. 2), but it is to be appreciated that the technology can be applied to other mutant genes and corresponding types of muscular dystrophy as well.

In one embodiment, an iPSC is subjected to CRISPR gene editing by introduction of a Cas9 protein, a guide RNA specific to the CAPN3 locus, and a template containing optimized CAPN3 coding sequences to be inserted downstream of the endogenous CAPN3 promoter. A nucleic acid sequence that encodes a normal CAPN3 protein is provided in GenBank Accession No: NM_000070.3.

In some embodiments, the endogenous CAPN3 gene contains one or more mutations such that the gene is incapable of expressing a functional CAPN3 protein. If the mutations are not in exon 1 or the first a few exons, then the first a few exons do not need to be replaced by the exogenous coding sequence.

Further, the instant inventors discovered that intron 1 of the CAPN3 gene is important for the expression of functional CAPN3 protein. Accordingly, in one embodiment, the exogenous CAPN3 coding sequence does not need to include exon 1 or even exon 2, and the insertion locus is after exon 2 of the endogenous exon 2 of the CAPN3 gene. In some embodiments, the CAPN3 coding sequence to be inserted starts with exon 3 (e.g., includes exons 3-24), and the insertion is at a locus that immediately follows (3′ to) the last nucleotide of exon 2 of the endogenous gene.

In some embodiments, the insertion is at a locus that is further downstream of exon 2, such as in intron 2, exon 3, intron 3, or exon 4, without limitation, so long as the retained exons do not include a mutation. The corresponding exogenous coding sequence would include the downstream exons to be complementary to the exons upstream of the insertion locus.

In an alternative embodiment, the CAPN3 coding sequence can be inserted into the target genome at a locus that is different from the endogenous CAPN3 gene, such as a “genomic safe harbor” (GSH). A genomic safe harbor (GSH) is a site in the genome able to accommodate the integration of new genetic material in a manner that ensures that the newly inserted genetic elements: (i) function predictably and (ii) do not cause alterations of the host genome posing a risk to the host cell or organism. A known GSH is the human AAVS1 gene (PPP1R12C; Ensembl ID ENSG00000125503).

Other GSHs, and methods for identifying new GSHs are known as well. For instance, in Aznauryan et al., Cell Rep Methods. 2022 Jan. 14;2(1):100154, the authors reported discovery of some new GSHs, including GSHI (GRCh38 coordinates 195,338,589-195,818,588), GSH2 (GRCh38 coordinates 22,720,711-22,761,389), GSH7 (GRCh38coordinates 145,090,941-145,219,513), GSH8 (GRCh38 coordinates 145,320,384-145,525,881), and GSH31 (GRCh38 coordinates 89,174,426-89,179,074).

When the exogenous CAPN3 coding sequence is inserted at a locus different from the endogenous gene, it cannot utilize the native CAPN3 promoter. In some embodiments, therefore, the exogenous CAPN3 coding sequence is operatively connected with a promoter that is capable of expressing the CAPN3 gene. In some embodiments, the expression is in a muscle cell. In some embodiments, the promoter is a muscle creatine kinase promoter (tMCK). tMCK is comprised of core sequences from the endogenous muscle creatine kinase (Ensembl ID ENSMUSG00000030399) promoter. It was generated by ligating a triple tandem repeat of the MCK enhancer to its basal promoter, thus generating a strong, muscle-specific promoter.

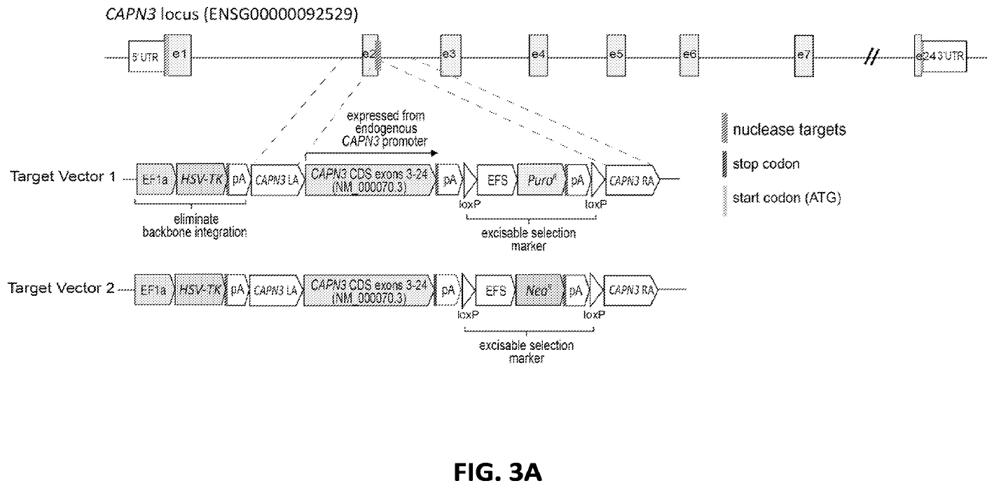

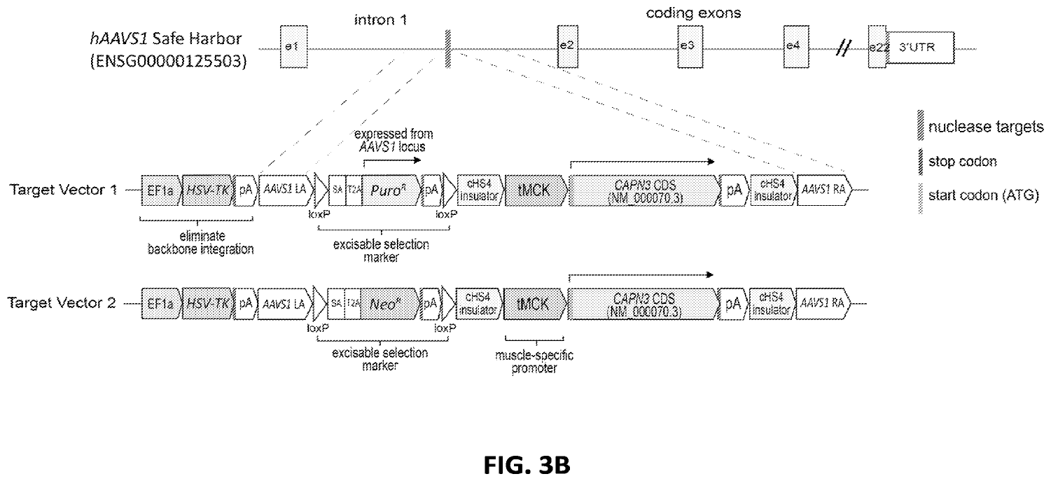

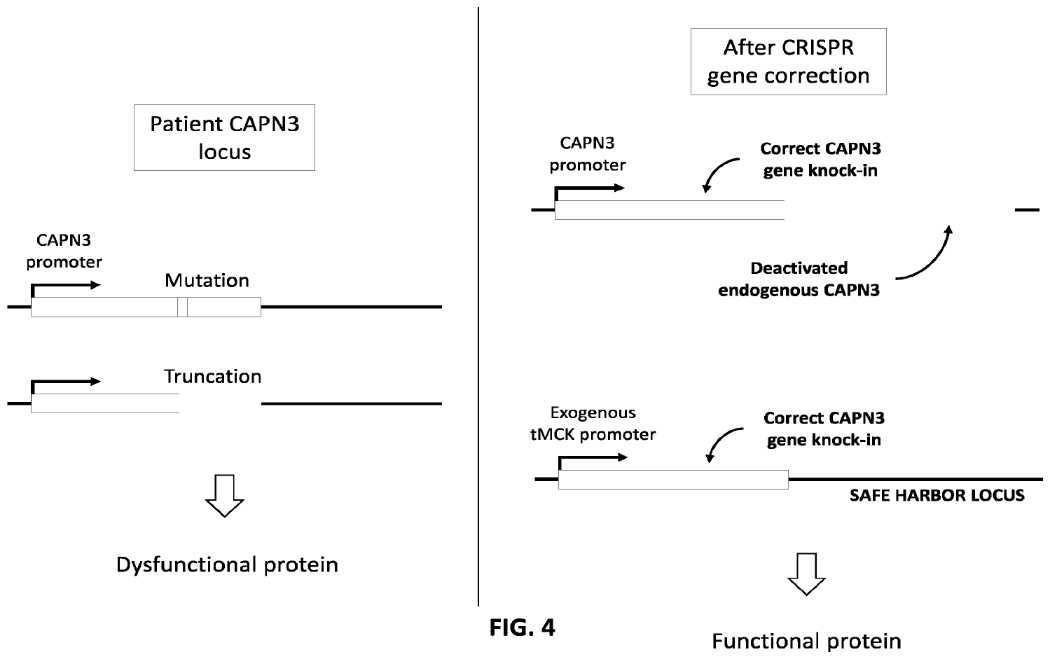

In some embodiments, the CAPN3 coding sequence is followed (after a poly-A tail) by a selection marker. In one embodiment, the selection marker is a puromycin or neomycin resistance gene, driven by a constitutive promoter (e.g., PGK (3-phosphoglycerate kinase) promoter). In some embodiments, it is preferred that both alleles of the mutant CAPN3 gene are corrected; accordingly, two copies of the exogenous CAPN3 coding sequences are integrated into the genome of the target iPSC. This can be achieved by transfecting two vectors each containing a CAPN3 coding sequence, but with a different selection marker (see, e.g., FIG. 3A-B). For instance, one of the vectors includes a puromycin resistance gene and the other includes a neomycin resistance gene. In addition to resistance genes to puromycin and neomycin, those resistant to others, such as gentamicin, blasticidin, and Zeocin, can also be used.

Upon transfection of two vectors, an iPSC that has integrated both copies of the CAPN3 coding sequence can be identified with both selection conditions, e.g., culture media with both puromycin and neomycin added (see, e.g., step 2 in FIG. 2).

In each vector, the to-be-integrated sequences can be flanked by CAPN3 gene specific homology arms to enable target-specific editing. For each selection marker, the expression cassette can be flanked by site-specific recombinase target sites to enable removal (e.g., loxP sites to allow Cre excision) of the cassettes (see, e.g., step 3 in FIG. 2) after the drug selection is completed.

In some embodiments, it is desirable to remove incorrectly integrated sequences. In one example, a constitutively expressed kill switch (e.g., a thymidine kinase gene cassette) is included in the vector, but is located outside of the CAPN3 gene homology arms. If the CAPN3 cassette is correctly integrated into the target genome, the kill switch is necessarily removed during the homology-orientated integration. If the kill switch is not removed, it means that the integration is incorrect. In such a situation, the cell can be killed by the corresponding kill switch drug treatment (e.g., ganciclovir).

In one example, after puromycin and G418 (a neomycin analog) selection, the cells are treated with ganciclovir. The resulting iPSC are then expanded and subjected to a round of treatment with TAT-Cre enzymes to remove the puromycin/neomycin selection cassette (which contains a Cre site), leaving only the CAPN3 coding sequence insert in place in the genome.

In some embodiments, the iPSCs that remain after the Cre-lox excision are expanded at low culture density for clonal isolation. Isolated clones are tested for the presence of inserted CAPN3 coding sequences on both copies of the genome, the absence of puromycin/neomycin selection cassettes, and the absence of randomly integrated vector backbones to create a master cell bank (MCB). The cells may be expanded and cryopreserved for later use (see, e.g., step 4 in FIG. 2).

For CAPN3 locus repair, for instance, the original mutant CAPN3 (exons 3-24) coding sequence is preceded by a newly integrated, exogenous CAPN3 (exons 3-24) coding sequence (FIG. 3A). The endogenous CAPN3 promoter drives expression of the exogenous CAPN3 coding expression, while the endogenous/mutant CAPN3 is inactivated (e.g., by the poly(A) tail/stop codon from the inserted cassette).

The process of inserting or incorporating a nucleic acid into a cell can be via known technologies, such as transformation, transfection or transduction, without limitation. Transformation introduces recombinant plasmid DNA into competent cells that take up extracellular DNA from the environment. This process is adapted for propagation of plasmid DNA, protein production, and other applications. Transfection is the process of uptake of foreign nucleic acid by a eukaryotic cell. Transduction refers to the introduction of a recombinant viral vector particle into a target cell.

The term “vectors” refers to a nucleic acid molecule capable of transporting or mediating expression of a heterologous nucleic acid. A plasmid is a species of the genus encompassed by the term “vector.” A vector typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA molecules which, in their vector form are not bound to the chromosome, and typically comprise entities for stable or transient expression or the encoded DNA. Other expression vectors that can be used in the methods as disclosed herein include, but are not limited to plasmids, episomes, bacterial artificial chromosomes, yeast artificial chromosomes, bacteriophages or viral vectors, and such vectors can integrate into the host's genome or replicate autonomously in the cell. A vector can be a DNA or RNA vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

In vitro Differentiation of CAPN3-Corrected iPSC

The CAPN3 gene-corrected iPSC can be further cultured, differentiated, enriched, and expanded to produce satellite cells.

In some embodiments, the iPSC are plated as single cells for cell culturing by adhesion culture without the use of feeder cells. For the culture, a culture vessel is used such as a dish, a flask, a microplate, or a cell culture sheet such as OptiCell (Nalge Nunc International).

In some embodiments, the culture vessel is surface-treated for improving adhesiveness to cells (hydrophilicity) or coated with a substrate for cell adhesion such as collagen, gelatin, poly-L-lysine, poly-D-lysine, laminin, fibronectin, extracellular matrix ((e.g., BD Matrigel (Becton Dickinson), Geltrex (Gibco)) or vitronectin. The culture vessel can be coated with type I collagen, Matrigel, fibronectin, vitronectin or poly-D-lysine. Culturing media includes use of mouse embryonic fibroblast-conditioned media. In some embodiments, the vessel is coated with a suitable adhesion molecule. Non-limiting examples of adhesion molecules include laminin-511,-521,-332 and -111, and fragments thereof. Laminin-511 is a major component of the basement membrane used as a scaffold for pluripotent stem cells (ES/iPS cells) as it binds to integrin on cell surfaces. Laminin-511 is a large protein (about 800 kDa) composed of three chains (α-, β-, and γ-) forming a supramolecular aggregate. Besides the whole protein, its fragments can also be suitably used. One such fragment is the E8 fragment (Miyazaki et al., Nature Commun. 3; 1236 (2012)). Recombinant laminin-511 E8 is commercially available as iMatrix 511.

In certain embodiments, the iPSC are cultured in media that includes a growth factor, e.g. fibroblast growth factor 2 (FGF-2) and/or a ROCK inhibitor e.g., Y-27632. The term “ROCK inhibitor” means a substance inhibiting Rho kinase (ROCK: Rho-associated, coiled-coil containing protein kinase) and may be substance inhibiting any of ROCK I and ROCK II. The ROCK inhibitor is not particularly limited as long as the ROCK inhibitor has the function described above. Examples of the ROCK inhibitor that can be used include: N-(4-pyridinyl)-4B-[(R)-1-aminoethyl]cyclohexane-1α-carboxamide (Y-27632), fasudil (HA1077), (2S)-2-methyl-1-[(4-methyl-5-isoquinolinyl]sulfonyl] hexahydro-1-H-1,4-diazepine (H-1152), 4B-[(1R)-1-aminoethyl]-N-(4-pyridyl)benzenecarboxamide (Wf-536),N-(1H-pyrrolo[2,3-b]pyridin-4-yl)-4PER(R)-1-aminoethyl]cyclohexane-carbox-amide (Y-30141), N-(3-{[2-(4-amino-1,2,5-oxadiazol-3-yl)-1-ethyl-1H-imidazo[4,5-c]pyridin-6-yl]oxy}phenyl)-4-{[2-(4-morpholinyl)ethyl]-oxy}benzamide (GSK269962A) and N-(6-fluoro-1H-indazol-5-yl)-6-methyl-2-oxo-4-[4-(trifluoromethyl)phenyl]-3,4-dihydro-1H-pyridine-5-carboxamide (GSK429286A); antibodies (including functional fragments), antisense nucleic acids, and siRNA against ROCK; ROCK antagonists and dominant negative forms; and other ROCK inhibitors known in the art.

In some embodiments, the culture media includes a base stem cell media and an N2 supplement media. The N2 media may include ingredients such as bovine or recombinant human insulin, transferrin, putrescine, selenite, and progesterone. In some embodiments, the base stem cell media and the N2 media are used in a ratio of 20:80 to 45:55 (v/v), or 30:70 to 45:55, 35:65 to 45:55 or about 40:60 (v/v).

In a coated vessel with suitable stem cell culture media, the iPSC can be cultured to reach a suitable target confluency (e.g., at least 40%, 50%, 60%, 70%, 80% or 90%, which can be determined by microscopy) is achieved. The iPSC are then subjected to differentiation.

The differentiation media, in one embodiment, includes a Wnt signaling activator (e.g., CHIR99021 (6-[[2-[[4-(2,4-dichlorophenyl)-5-(4-methyl-1H-imidazol-2-yl)-2-pyrimidin-yl]amino]ethyl]amino]nicotinonitrile)), a ROCK inhibitor (e.g., Y-27632 (N-(4-pyridinyl)-4B-[(R)-1-aminoethyl]cyclohexane-1α-carboxamide)), a Notch signaling inhibitor (e.g., Y-27632), a TGF-β signaling inhibitor (e.g., SB431542 (4-[4-(2H-1,3-Benzodioxol-5-y1)-5-(pyridin-2-yl)-1H-imidazol-2-yl]benzamide)) and/or N2 media, in various combinations (see, e.g., Table 2).

CHIR99021 (6-[[2-[[4-(2,4-dichlorophenyl)-5-(4-methyl-1H-imidazol-2-yl)-2-pyrimidin-yl]amino]ethyl]amino]nicotinonitrile) is a Wnt signaling activator, as well as a GSK3β inhibitor. GSK3β (glycogen synthase kinase 3) is a serine/threonine protein kinase which participates in various signal pathways involved in the production of glycogen, apoptosis, the maintenance of stem cells. GSK3 includes two isoforms, α and β. Examples of the GSK3β inhibitor include CHIR98014 (2-[[2-[(5-nitro-6-aminopyridin-2-yl) amino]ethyl]amino]-4-(2,4-dichloroph-enyl)-5-(1H-imidazol-1-yl)pyrimidine), CHIR99021 (6-[[2-[[4-(2,4-dichlorophenyl)-5-(4-methyl-1 H-imidazol-2-yl)-2-pyrimidin-yl]amino]ethyl]amino]nicotinonitrile), Kenpaullone, AR-A0144-18, TDZD-8 (4-benzyl-2-methyl-1,2,4-thiadiazolidine-3,5-dione), SB216763 (3-(2,4-dichlorophenyl)-4-(1-methyl-1H-indol-3-yl)-1H-pyrrole-2,5-dione),BIO (6-bromoindirubin-3-oxime), TWS-119(3-[6-(3-aminophenyl)-7H-pyrrolo[2,3-d]pyrimidin-4-yloxy]phenol) and SB415286 (3-[(3-chloro-4-hydroxyphenyl) amino]-4-(2-nitrophenyl)-1H-pyrrol-e-2,5-dione). Also, an antisense oligonucleotide, siRNA, or the like against GSK3β mRNA can be used as the GSK3β inhibitor and is commercially available or can be synthesized according to a method known in the art.

The concentration of the Wnt signaling activator/GSK3β inhibitor (e.g., CHIR99021), in some embodiments, may be at least 0.2 μM, 0.5 μM, 1 μM, 1.5 μM, 2 μM, 2.5 μM, or 3 μM. The concentration, in some embodiments, may not be higher than 3 μM, 3.5 μM, 4 μM, 4.5 μM, 5 μM, 6 μM, 7 μM, 8 μM, 9 μM, 10 μM, 15 μM, 20 μM, 30 μM or 40 μM. The concentration, in some embodiments, may be 0.2-10 μM, 0.5-8 μM, 1-7 μM, 2-5 μM, or 2-4 μM, without limitation.

In some examples, the iPSC are cultured with a γ-secretase inhibitor and/or Notch signaling inhibitor. An example of a γ-secretase inhibitor is N-[N-(3,5-Difluorophenacetyl)-L-alanyl]-S-phenylglycine t-butyl ester (DAPT). DAPT is a potent and specific inhibitor of γ-secretase that blocks Notch signaling, a multimeric membrane protein complex that catalyzes proteolytic cleavage of amyloid precursor protein (APP) resulting in the accumulation of amyloid-β (Aβ) peptides which is associated with early on-set of familial Alzheimer's disease (AD). It directly binds to the C-terminal fragment of the catalytic center of γ-secretase, presenilin (PS), especially within the transmembrane domain 7 or more C-terminal region, resulting in the synthesis of a photoactivable DAPT derivative. DAPT indirectly inhibits Notch, which is a substrate for γ-secretase.

A Notch signaling inhibitor is an agent, e.g., a chemical compound or an antibody, that inhibits the Notch signaling pathway. Inhibitors to the γ-secretase, for instance, can inhibit the Notch signaling pathway. Such a γ-secretase inhibitor is for example peptidic in nature or non-peptidic or semi-peptidic and is preferably a small molecule. Examples include DAPT (N-[N-(3,5-difluorophenylacetyl)-L-alanyl]-S-phenylglycine t-butyl ester). Also compounds from the chemical classes AS (arylsulfonamide), DBZ (dibenzazepine (DBZ), BZ (benzodiazepine), LY-411,575 and many others, have been tested for their γ-secretase inhibiting activity. The γ-secretase inhibitors have been divided in solfonamides/sulfones and benzodiazepines/benzolactams.

In certain embodiments, the iPSC are cultured in media comprising at least a Notch signaling inhibitor (e.g., DAPT) for at least about 1 day, or for at least about two days, or for at least about three days, or for at least about four days, of for at least about five days, or for at least about 6 days, or for at least about seven days, or for at least about eight days. In certain embodiments, the cells are cultured in media comprising at least a Notch signaling inhibitor (e.g., DAPT) for 1 to 20 days, 2 to 15 days, 3 to 12 days, 4 to 11 days, 5 to 10 days, 6 to 9 days, 7 to 9 days, 7-8 days, or 8-10 days, without limitation.

In some embodiments, the media includes a TGF-β inhibitor. TGF-β inhibitors include, for example, SB431542 (4-[4-(2H-1,3-Benzodioxol-5-yl)-5-(pyridin-2-yl)-1H-imidazol-2-yl]benzamide), SB202190 (Lindemann et al., Mol. Cancer, 2003, 2:20), SB505124 G (GlaxoSmithKline), Lefty-1 (e.g., NM_020997), NPC30345, SD093, SD908, SD208 (Scios), LY2109761, LY364947, LY580276 (Lilly Research Laboratories), A-83-01 (WO2009146408) and their derivatives.

The concentration of TGF-β inhibitor in the medium, in some embodiments, is 1 μM to 50 μM, for example, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM 16 μM, 17 μM, 18 μM, 19 μM, 20 μM, 25 μM, 30 μM, 35 μM, 40 μM, 45 μM, and 50 μM. In some embodiments, the concentration is 2 μM to 10 μM, such as 5 μM.

In an example procedure, the iPSC differentiation is carried out as shown in Table 2. On each specified day, the media is completely exchanged with the indicated supplements (e.g., Wnt signaling activator) added.

TABLE 2
Example iPSC Differentiation Procedure

| Day | Fresh media (with supplements as indicated): |
|---|---|
| 0 | Wnt signaling activator + N2 media |
| 1 | |
| 2 | Wnt signaling activator + N2 media |
| 3 | |
| 4 | Notch signaling inhibitor + N2 media |
| 5 | |
| 6 | Notch signaling inhibitor + TGF-β inhibitor + N2 media |
| 7 | |
| 8 | Notch signaling inhibitor + TGF-β inhibitor + N2 media |
| 9 | |
| 10 | Notch signaling inhibitor + TGF-β inhibitor + N2 media |
| 11 | |
| 12 | |
| 13 | |
| 14-30 | N2 media (exchanged every other day) |

### Sorting and Expansion

The differentiated cells can be sorted by magnetic assisted cell sorting (MACS). In some embodiments, the sorted cells are identified with an antibody that binds CD271, which can be coupled to magnetic beads.

CD271 belongs to the low-affinity neurotrophin receptors and the tumor necrosis factor receptor superfamily. Initially the human CD271 (LNGFR) was described to be expressed on cells of the central and peripheral nervous system, and was suggested to be involved in the development, survival, and differentiation of cells. It is discovered herein that CD271 is a suitable marker to identify differentiated satellite cells suitable for treatments.

The sorted cells can be expanded in a suitable media, such as the N2 media supplemented with 10% FBS, bFGF2, and FGF8b, for 2-3 weeks.

The enriched and expanded CD271+ satellite cells have excellent repopulation and engraftment capabilities. Also the regeneration and engraftment capability of the CD271+ satellite cells requires a minimum cell concentration.

In accordance with one embodiment of the present disclosure, therefore, provided is a population of cells (e.g., mammalian cells, or more particularly human cells) wherein at least 30% of the cells are CD271+ satellite cells derived from iPSC in vitro or ex vivo. In some embodiments, the cell population includes at least 35%, 40%, 45%, 50%, 55%, 60%, 65%, 70%, 75%, 80%, 85%, 90% or 95% CD271+ satellite cells.

In some embodiments, a cell population includes at least 100, 1000, 10,000, 100,000, 1×10⁶, 1×10⁷, 1×10⁸, or 1×10⁹ cells. In some embodiments, a substantial portion of the cells of the population have been cultured along with the CD271+ satellite cells during the differentiation. For instance, at least 50%, 60%, 70%, 80%, 90%, 95%, 98%, 99%, 99.5%, or 99.9% of the cells of the population have been cultured along with the CD271+ satellite cells during the differentiation.

In addition to CD271+, the differentiated cells can also be characterized as NCAM⁺, HNK1⁻, CD271+, MyoD+, CD54+, integrin α9β1+ and/or SDC2+. Moreover, suitable markers for identifying these cells include, without limitation, CHRNA1+, NTSR1+, FZD1+,FZD5−, GPR37− and GPR27−. In some embodiments, the differentiated CD271+ satellite cells are characterized by at least two, three, four, or five of CHRNA1+, NTSR1+, FZD1+, FZD5−, GPR37− and GPR27−.

Whether a cell surface protein is positive (+) or negative (−) can be assessed by agents recognizing the marker, such as an antibody. It is readily appreciated by the skilled artisan, however, such positive and negative may not be absolute. In some embodiments, a marker being positive is to have a higher expression on the cell than on a reference cell. In some embodiments, a marker being negative is to have a lower expression on the cell than on a reference cell. The reference cell, for instance, is a cell likewise differentiated from a pluripotent stem cell but cannot regenerate a muscle tissue in vivo.

The produced cells, in some embodiments, can be cryopreserved for later use.

### Treatments

Compositions, uses, therapies, medicaments and methods are also provided for treating Limb-girdle muscular dystrophy Type 2A (LGMD2A).

The satellite cells can be administered to a patient as a pharmaceutically or physiologically acceptable preparation or composition containing a physiologically acceptable carrier, excipient, or diluent, and administered to the tissues of the recipient organism of interest, including humans and non-human animals.

The satellite cell composition can be prepared by resuspending the cells in a suitable liquid or solution such as sterile physiological saline or other physiologically acceptable injectable aqueous liquids. The amounts of the components to be used in such compositions can be routinely determined by those having skill in the art.

In examples, for injectable administration, the composition (e.g., a composition comprising satellite cells) is in sterile solution or suspension or can be resuspended in pharmaceutically-and physiologically-acceptable aqueous or oleaginous vehicles, which may contain preservatives, stabilizers, and material for rendering the solution or suspension isotonic with body fluids (i.e., blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

Consistent with the present invention, the satellite cells can be administered to body tissues, including muscle. The number of satellite cells in a suspension and the mode of administration may vary depending on the site and condition being treated.

In embodiments, a therapeutically effective amount of the composition (e.g., a composition comprising satellite cells) in humans can be administered. In one embodiment, the composition (e.g., a composition comprising satellite cells) is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In an embodiment, the composition (e.g., a composition comprising satellite cells) is administered once a week, or once every two weeks, or once every 3 weeks or once every 4 weeks for at least 1 week, in some embodiments for 1 to 4 weeks, from 2 to 6 weeks, from 2 to 8 weeks, from 2 to 10 weeks, or from 2 to 12 weeks, 2 to 16 weeks, or longer (e.g., 1, 2, 3, 4, 5, 6, 7, 8, 9, 10, 11, 12, 36, 48, or more weeks).

### EXPERIMENTAL EXAMPLES

### Example 1. Insertion of Exogenous CAPN3 Coding Sequences

This example designed and tested the insertion of exogenous CAPN3 coding sequences into a target human genome to enable expression of an active CAPN3 protein.

Target vectors encoding the human CAPN3 coding sequence (CDS) were constructed as double-stranded DNA plasmids and designed to integrate into specific genomic loci using a DNA-cleaving nuclease. The encoded CAPN3 CDS nucleotide sequence was defined by the NCBI reference sequence NM_000070.3, the transcription of which is terminated by a transgenic polyadenylation signal (pA) after the stop codon of the inserted CAPN3 CDS.

Each target vector contains a loxP-flanked, excisable drug selectable marker (PuroR and NeoR) expressed by an internal promoter (EFS; Elongation Factor 1a short) (FIG. 3A) or by the endogenous hAAVS1 locus in which the selectable marker is spliced into the translational reading frame of the hAAVS1 CDS via a splice (SA) and self-cleaving peptide sequence (T2A) (FIG. 3B). The selectable marker can be excised from the genome, after integration of the target vector, by treatment of the cells with Cre recombinase.

The regions of the vector intended to incorporate into the genome are flanked by homology arms 500-100 nucleotides in length (LA; Left Arm, RA; Right Arm), which are homologous to the regions of the genome flanking where the insertion will occur. Short sequences in the genome, positioned between homology arms, define the site where the nuclease cleaves the DNA in the presence of an RNA oligonucleotide that is complimentary to the genomic nuclease target site. The double stranded break induces the cell to integrate the regions of the target vectors flanked by the homology arms into the genome. Each target vector encodes the herpes simplex virus 1 thymidine kinase (HSV-TK) suicide gene expressed by an upstream promoter (EF1a; Elongation Factor 1a full). This allows for elimination of the cells, using the prodrug ganciclovir, that integrate vector backbone elements, which are not contained within the homology arms, into the cell.

Two integration strategies were tested here. In the first, as illustrated in FIG. 3A, exons 3-24 of the CAPN3 CDS encoded by the target vectors were inserted, in frame, after the last nucleotide of exon 2 from the endogenous CAPN3 gene (Ensembl ID; ENSG00000092529). The inserted CAPN3 exons 3-24 were therefore transcribed as part of the final CAPN3 mRNA, which included the endogenous exons 1 and 2, under control of the endogenous CAPN3 promoter. The entire transcript was terminated by a transgenic pA signal encoded in the target vector.

In the second strategy, as illustrated in FIG. 3B, all of exons 1-24 of the entire CAPN3 CDS were inserted into intron 1 of the AAVS1 gene (PPP1R12C; Ensembl ID ENSG00000125503). The CAPN3 CDS is expressed under control of a synthetic muscle specific promoter (tMCK), which is comprised of core sequences from the endogenous muscle creatine kinase (Ensembl ID ENSMUSG00000030399) promoter. The promoter, CAPN3 CDS, and pA sites are flanked by minimal Chicken hypersensitive site 4 (cHS4) insulators, which mitigate transcriptional silencing of the CAPN3 CDS.

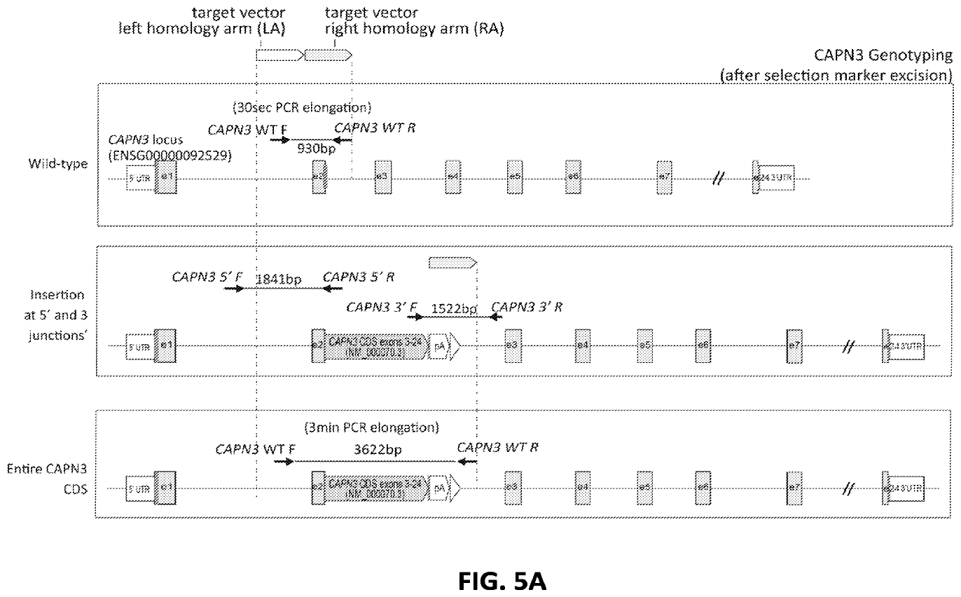

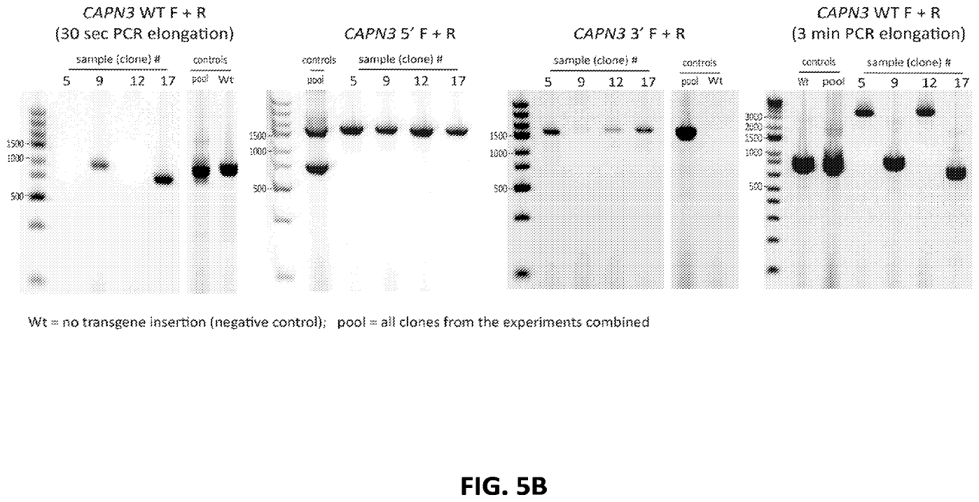

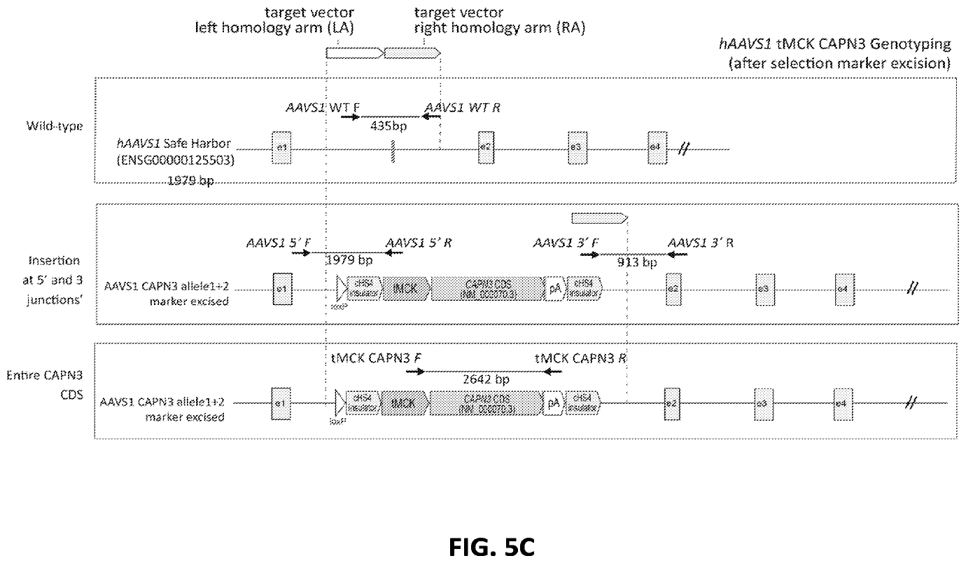

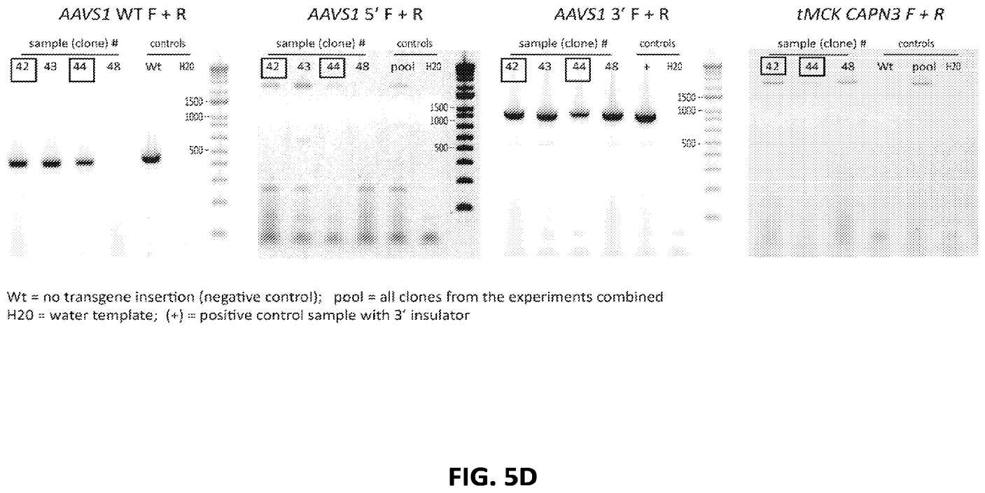

Genotyping was used to confirm integration of the exogenous CAPN3 coding sequences at the designated locations. For the first and second integration strategies (FIG. 3A and 3B, respectively), the genotyping designs are illustrated in FIG. 5A and 5C, respectively. As shown in FIG. 5A, the PCR primers were so selected that the amplified sequence from the integrated site would be 1522 bp in length (middle panel), and that from the wild-type would be 3622 bp in length.

More specifically, in FIG. 5A, boxes showing target vector left and right homology arms (not drawn to scale) indicate position relative to genotyping primers. Cells were electroporated with the CAPN3-targeting vectors, Cas9 nuclease, and RNA oligonucleotides that were complimentary to a region at the end endogenous exon 2 of the CAPN3 gene. Cells without incorporation of the target vectors were eliminated by addition of antibiotics to the cell culture after electroporation (Puromycin and G418; resistance encoded by the PuroR and NeoR transgenes on the target vectors). Antibiotic-resistant cells were treated with Cre recombinase (TAT-Cre) to excise the loxP-flanked selectable markers. Cells were then sparse plated such that clonal, single-cell derived colonies could be isolated and expanded for genotyping.

A series of polymerase chain reactions was performed on genomic DNA purified from each clonal line to determine correct insertion. First, primers (CAPN3 WT F+R, 30 sec elongation) to detect an endogenous CAPN3 allele without integration were run to distinguish clones with hemizygous (1 copy) and homozygous (2 copy) integration of the target vector(s). Then, primers were used to amplify the 5′ and 3′ regions of the genome-insert junction (CAPN3 5′ F+R and CAPN3 3′ F+R, respectively). In the 5′ reaction, the forward primer is outside the genomic region encoded by left homology arm, and the reverse primer is inside the target vector. In the 3′ reaction, the forward primer is inside the target vector and the reverse primer is outside the genomic region encoded by the right homology arm. Finally, The CAPN3 WT F+R primers were used at 3 min PCR elongation time to amplify the entire inserted CAPN3 CDS exons 3-24.

All PCR reactions were analyzed by gel electrophoresis (FIG. 5B) to verify clones with the expected amplification of each band for all reactions, the size of which was predicted based on the location of the primer binding in the genome. Clones 5 and 12 were homozygous clones (2 copies); clones 9 and 17 were hemizygous (1 copy).

As shown in FIG. 5C, cells were electroporated with hAAVS1-targeting vectors, Cas9 nuclease, and RNA oligonucleotides that were complimentary to a region of intron 1 in the hAAVS1 locus. Cells without incorporation of the target vectors were eliminated by addition of antibiotics to the cell culture after electroporation (Puromycin and G418; resistance encoded by the PuroR and NeoR transgenes on the target vectors). Antibiotic-resistant cells were treated with Cre recombinase (TAT-Cre) to excise the loxP-flanked selectable markers. Cells were then sparse plated such that clonal, single-cell derived colonies could be isolated and expanded for genotyping.

A series of polymerase chain reactions was performed on genomic DNA purified from each clonal line to determine correct insertion. First, primers (AAV1 WT F+R) to detect an AAVS1 wild-type allele without integration were run to distinguish clones with hemizygous (1 copy) and homozygous (2 copy) integration of the target vector(s). Then, primers were used to amplify the 5′ and 3′ regions of the genome-insert junction (AAVS1 5′ F+R and AAVS1 3′ F+R, respectively). In the 5′ reaction, the forward primer is outside the genomic region encoded by the left homology arm, and the reverse primer is inside the target vector. In the 3′ reaction, the forward primer is inside the target vector and the reverse primer is outside the genomic region encoded by the right homology arm. Finally, primers were used to amplify the entire CAPN3 CDS (tMCK CAPN3 F+R), in which the forward primer is inside the tMCK promoter, and the reverse primer is inside the pA sequence.

All PCR reactions were analyzed by gel electrophoresis (FIG. 5D) to verify clones with the expected amplification of each band for all reactions, the size of which was predicted based on the location of the primer binding in the genome. Boxes around clones 42 and 44 indicate these clones have correct, hemizygous (1 copy) insertion of the CAPN3-encoding target vector.

### Example 2. Expression of Active CAPN3 Protein in Engineered Cells

This example checked whether human cells engineered to integrate exogenous CAPN3 coding sequences can express functional CAPN3 mRNA and protein.

### Material and Methods

### Myotube Formation

iPSC derived satellite cells were seeded at 3×10⁴ cells cultured in the growth medium (N2 medium+10% FBS+FGF2+FGF8) for 2 days and the medium was switched to N2 medium supplemented with 10 μM TGFb inhibitor (SB431542), 10 μM Forskolin, 10 μM DAPT, 10 μM Dexamethasone and cells were cultured for 5 additional days to form matured myotubes.

### QPCR

Cells were lysed in buffer RLT (RNasey mini kit, Qiagen) and homogenized using QIAshredder. RNA was extracted from the lysate using RNasey mini kit (Qiagen) with on-column DNAse treatment following manufacturer's instructions. RNA concentration was quantified using Nanodrop. For quantitative RT-PCR analysis, reverse transcription was performed using iScript RT Supermix (bio-Rad). qPCR was performed using taqman probes (Thermo Scientific) and TaqMan Fast Advanced Master Mix. QPCR was performed using QuantStudio™ 5 Real-Time PCR System. Taqmen probes used are CAPN3 (Hs01115989_m1), GAPDH (Hs99999905_m1), ACTB (Hs99999903_m1).

### Western Blot Analysis

Cells were lysed in lysis buffer consisting of 20 mM Tris HCl, 0.1 mM EDTA, 1 mM DTT, 20 mg/mL soybean trypsin inhibitor, 28 mM E64, and 2 mM phenylmethylsulfonyl fluoride (PMSF) plus 1xLaemmli sample buffer. The lysate was collected using cell scraper, boiled at 95° C. for 5 min, and centrifuged for 10 min at 10,000 rpm to remove cell debris. Total protein concentration was measured using a Bradford assay. A total of 30 mg protein was electrophoresed in 10% SDS-PAGE gels. After electrophoresis, proteins were transferred to nitrocellulose via Transblot Turbo Transfer system. After transfer, the blot was blocked with 5% BSA in PBS-T (Phospho-buffered saline and 0.1% Tween 20) for 1 h at room temperature (RT). After the blocking, the blot was incubated with primary antibody (CAPN3: NCL-CALP-12A2;Desmin: 5332S; GAPDH: 2118S) diluted in 3% BSA in PBS-T and incubated at 4° C. overnight. The next day, the membranes were incubated with secondary antibodies and imaged with Li-cor Odyssey images.

### Results

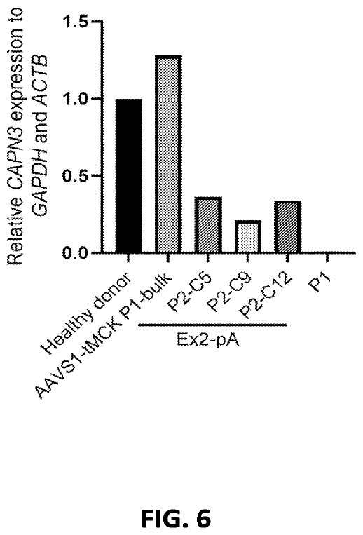

qPCR was used to detect relative CAPN3 mRNA in the engineered cells (see Example 1). The results are shown in FIG. 6.

The CAPN3 mRNA level in myofibers from a healthy donor was used as control (=1.0). Among all samples, the one in which the exogenous complete CAPN3 coding sequence was inserted (patient 1 (P1): AAVS1-tMCK P1-bulk) had the highest expression. All three clones (5, 9 and 12) with corrected CAPN3 in the CAPN3 locus exhibited high expression levels as well (patient 2 (P2). By contrast, in myofibers with uncorrected CAPN3,no functional mRNA was detected (P1 without correction).

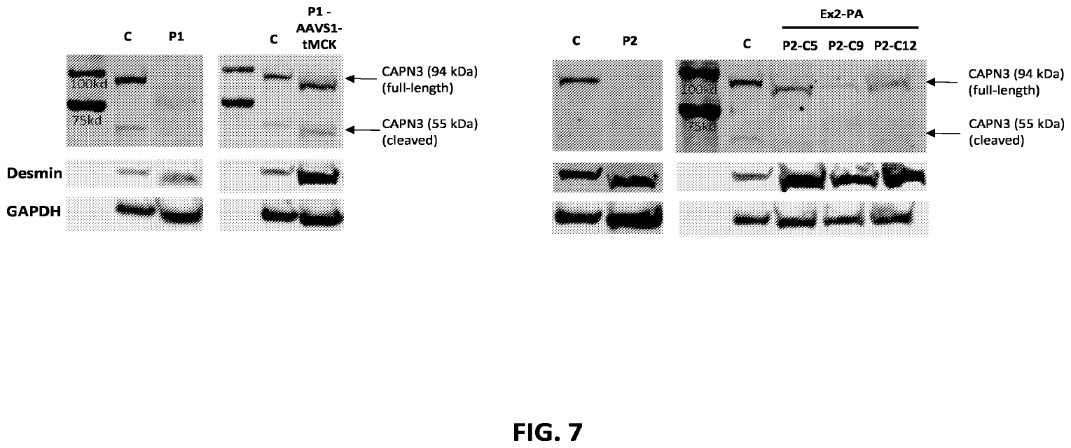

Western blots were used to detect CAPN3 protein levels in these myofibers. As shown in FIG. 7, left panel (P1), while the control (C, from healthy donor) and AAVS1-inserted samples showed positive CAPN3 expression, the P1 without correction showed no expression. Likewise, in FIG. 7, right panel, the control (C) and three corrected samples (in the CAPN3 locus) showed positive CAPN3 expression, while the un-corrected P2 did not.

This example, therefore, demonstrated that the instant technology of targeted insertion of exogenous CAPN3 coding sequences can restore functional expression of the CAPN3 protein.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for preparing a satellite cell from a stem cell comprising a mutation in an endogenous gene, comprising:
introducing to the stem cell a first and a second vectors, each comprising a first expression cassette encoding at least part of the wild-type gene, such that each of the first expression cassettes is integrated at a locus of the endogenous gene in the genome to enable coding of a wild-type version of the gene without the mutation;
differentiating the stem cell into a satellite cell; and
expanding the satellite cell into a population of satellite cells.

2. A method for preparing a satellite cell from a stem cell comprising a mutation in an endogenous gene, comprising:
introducing to the stem cell a first and a second vectors, each comprising a first expression cassette comprising all coding sequences of the wild-type gene, such that each of the first expression cassettes is integrated at a locus in the genome different from the endogenous gene;
differentiating the stem cell into a satellite cell; and
expanding the satellite cell into a population of satellite cells.

3. The method of claim 2, wherein the locus is selected from the group consisting of an intron of the AAVS1 gene, and human genome 195,338,589-195,818,588, 22,720,711-22,761,389, 145,090,941-145,219,513, 145,320,384-145,525,881, and 89,174,426-89,179,074 (GRCh38 coordinates).

4. The method of claim 3, wherein the locus is in intron 1 of the AAVS1 gene.

5. The method of claim 2, wherein the expression cassette further comprises a promoter capable to initiating expression of the coding sequences in a muscle cell.

6. The method of claim 5, wherein the promoter is a muscle creatine kinase promoter (tMCK).

7. The method of claim 1, wherein the mutation is a homozygous mutation.

8. The method of claim 1, wherein the stem cell is an induced pluripotent stem cell (iPSC) derived from a peripheral blood mononuclear cell or fibroblast isolated from a patient having muscular dystrophy.

9. The method of claim 8, wherein the muscular dystrophy is selected from the group consisting of limb-girdle muscular dystrophy, Becker muscular dystrophy, congenital muscular dystrophy, Duchenne muscular dystrophy, distal muscular dystrophy, Emery-Dreifuss muscular dystrophy, facioscapulohumeral muscular dystrophy, myotonic muscular dystrophy, and oculopharyngeal muscular dystrophy.

10. The method of claim 1, wherein the gene is selected from the group consisting of CAPN3, DNAJB6, TNPO3, HNRPDL, CAPN3, COL6A1, COL6A2, COL6A3, DYSF, SGCA, SGCB, SGCG, SGCD, TCAP, TRIM32, FKRP, TTN, POMT1, ANO5, FKTN, POMT2, POMGNT1, DAG1, PLEC1, TRAPPC11, GMPPB, ISPD, POGLUT1, COL6A1, COL6A2, COL6A3, LAMA2, POMGNT2, POPDC1, POPDC3, JAG2, PYROXD1, DMD, LAMA-2, DMD, DYSF, DMPK, CNBP, DUX4, PABPN1, EMD, LMNA,, SYNE1, SYNE2, FHL1, and TMEM43.

11. The method of claim 10, wherein the gene is CAPN3.

12. The method of claim 11, wherein the at least part of the wild-type CAPN3 gene does not include exon 1 or 2, and the locus is downstream of exon 2 of the endogenous CAPN3 gene.

13. The method of claim 12, wherein the at least part of the wild-type CAPN3 gene comprises exons 3-24, and the locus is 3′ to the last nucleotide of exon 2 of the endogenous CAPN3 gene.

14. The method of claim 1, wherein the first vector further comprises a second expression cassette encoding a first selection marker, and the second vector further comprises a third expression cassette encoding a second selection marker, wherein the first expression cassette and the second expression cassette on the first vector together are integrated into the genome, and the first expression cassette and the third expression cassette on the second vector together are integrated into the genome.

15. The method of claim 14, wherein the second and third expression cassettes are excised from the genome following confirmation of the integration.

16. The method of claim 1, wherein the first vector and second vector each further comprises a fourth expression cassette encoding a kill switch, which is not integrated to the loci.

17. The method of claim 1, further comprising, prior to differentiation, culturing the stem cell in a vessel coated with an adhesion molecule.

18. The method of claim 17, wherein the adhesion molecule is laminin-511 or a fragment thereof.

19-22. (canceled)

23. The method of claim 1, wherein the satellite cell is identified with an antibody specific to CD271.

24. (canceled)

25. The method of claim 1, further comprising administering the prepared satellite cell to a patient having the mutation.
